(19) United States
(12) Patent Application Publication
(10) Pub. No.: US 2011/0182848 A1
(43) Pub. Date: Jul. 28, 2011

(54) GRANULOCYTE COLONY STIMULATING FACTOR

(75) Inventors: Peter Artymiuk, Sheffield (GB); Richard Ross, Sheffield (GB); Jon Sayers, Sheffield (GB)

(73) Assignee: ASTERION LIMITED, Sheffield (GB)

(21) Appl. No.: 12/671,987

(22) PCT Filed: Jul. 31, 2008
(86) PCT No.: PCT/GB2008/002594

§ 371 Date: Apr. 7, 2011

Related U.S. Application Data
(60) Provisional application No. 60/956,303, filed on Aug. 16, 2007.

(30) Foreign Application Priority Data
Aug. 3, 2007 (GB) 0715133.5
Dec. 8, 2007 (GB) 0724013.8

(51) Int. Cl. A61K 38/19; C07K 14/535; C07H 21/04; C12N 15/63; C12N 5/16; A61P 37/04; C12N 1/00; C12N 5/14

(52) U.S. Cl. 424/85.1; 530/351; 536/23.4; 435/320.1; 435/325; 435/243; 435/419

(57) ABSTRACT

We disclose granulocyte colony stimulating factor fusion polypeptides; nucleic acid molecules encoding said polypeptides and methods of treatment that use said proteins.

The invention relates to granulocyte colony stimulating factor (GCSF) fusion polypeptides and dimers; nucleic acid molecules encoding said polypeptides and methods of treatment that use said proteins/dimers.

Cytokine receptors can be divided into three separate groups. Class 1 (referred to as the haemotopoietin or growth hormone family) receptors are characterised by four conserved cysteine residues in the amino terminal part of their extracellular domain and the presence of a conserved Trp-Ser-Xaa-Trp-Ser motif in the C-terminal part. The receptors consist of two polypeptide chains. Class I receptors can be sub-divided into the GM-CSF sub-family (which includes IL-3, IL-5, GM-CSF, GCSF) and IL-6 sub-family (which includes IL-6, IL-11 and IL-12). In the IL-6 sub-family there is a common tranduscing subunit (gp130) that associates with one or two different cytokine subunits. There is a further sub-family referred to as the IL-2 sub-family (includes IL-2, IL-4, IL-7, IL-9 and IL-15. The repeated Cys motif is also present in Class 2 (interferon receptor family) the ligands of which are α, α and γ interferons but lack the conserved Trp-Ser-Xaa-Trp-Ser motif.

GCSF stimulates the proliferation and differentiation of granulocyte progenitor cells. GCSF is encoded by a single gene that encodes two polypeptides that result from differential splicing of mRNA. The polypeptides are 177 and 180 amino acids in length with the mature polypeptide having a molecular weight of 19.6 kD. GCSF is produced by the endothelium and macrophages and acts through the GCSF receptor (GCSFR) which is expressed on granulocyte progenitor cells in bone marrow which when activated results in their maturation into granulocytes. These can then differentiate into neutrophil precursors and mature neutrophils. The main therapeutic application of recombinant GSCF is in the treatment of patients undergoing chemotherapy for cancer which results in the loss of neutrophils and consequently the development of neutropenia. Neutropenia results in immune suppression and exposure of the patient to infection and sepsis. In addition recombinant GCSF is used to increase the number of haematopoietic stem cells in vivo prior to harvesting and use in haematopoietic stem cell transplantation.

This disclosure relates to the identification of GCSF recombinant forms that have improved pharmacokinetics (PK) and activity. The new GCSF molecules have biological activity, form dimers and have improved stability.

According to an aspect of the invention there is provided a nucleic acid molecule comprising a nucleic acid sequence that encodes a polypeptide having the activity of granulocyte colony stimulating factor comprising a granulocyte colony stimulating factor polypeptide linked, directly or indirectly, to at least one cytokine binding domain of the granulocyte colony stimulating factor receptor polypeptide.

According to an aspect of the invention there is provided a fusion polypeptide comprising: the amino acid sequence of granulocyte colony stimulating factor polypeptide, or active part thereof linked, directly or indirectly, to at least one cytokine binding domain of the granulocyte colony stimulating factor receptor polypeptide.

In a preferred embodiment of the invention said fusion polypeptide comprises two cytokine homology binding domains of the granulocyte colony stimulating factor receptor polypeptide.

In a further preferred embodiment of the invention said fusion polypeptide further comprises an immunoglobulin-like domain.

In a further preferred embodiment of the invention said fusion polypeptide includes at least one fibronectin domain III; preferably two or three fibronectin III domains.

The GCSFR is complex comprises a series of domains that contribute to its molecular structure. GCSFR can be sub-divided into several regions that are structurally and functionally defined. The receptor is 812 amino acids in length and is typical of cytokine receptors in so far as it includes an extracellular domain, a single transmembrane domain and a cytoplasmic domain. The extracellular domain has a modular structure comprising from the amino terminus in the mature polypeptide; an immunoglobulin-like domain (amino acids 1-97); a first cytokine homology domain (97-201) and second cytokine domain (202-313) and three fibronectin III domains. Functionally the first and second cytokine domains bind GCSF.

In a preferred embodiment of the invention said fusion polypeptide comprises amino acid residues 97-201 as represented in SEQ ID NO: 31

In an alternative preferred embodiment of the invention said fusion polypeptide comprises amino acid residues 202-313 of SEQ ID NO: 31.

In an alternative preferred embodiment of the invention said fusion polypeptide comprises amino acid residues 97-313 of SEQ ID NO: 31.

In a further preferred embodiment of the invention said fusion polypeptide comprises amino acid residues 1-97 of SEQ ID NO: 31.

In a preferred embodiment of the invention polypeptide is linked to the cytokine binding domain wherein said granulocyte colony stimulating factor polypeptide is positioned amino terminal to said cytokine binding domain in said fusion polypeptide.

In an alternative preferred embodiment of the invention granulocyte colony stimulating factor polypeptide is linked to the cytokine binding domain wherein said granulocyte colony stimulating factor polypeptide is positioned carboxyl-terminal to said cytokine binding domain in said fusion polypeptide.

In a preferred embodiment of the invention granulocyte colony stimulating factor is linked to the binding domain of the granulocyte colony stimulating factor receptor polypeptide by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5, 6, 7, 8, 9 or 10 copies of the peptide Gly Gly Gly Gly Ser.

Preferably said peptide linking molecule consists of 6 copies of the peptide Gly Gly Gly Gly Ser.

In an alternative embodiment of the invention said polypeptide does not comprise a peptide linking molecule and is a direct fusion of granulocyte colony stimulating factor polypeptide and the binding domain of granulocyte colony stimulating factor receptor polypeptide.

According to an aspect of the invention there is provided a nucleic acid molecule comprising a nucleic acid sequence selected from:
i) a nucleic acid sequence as represented in SEQ ID NO:5;
ii) a nucleic acid sequence as represented in SEQ ID NO 7:;
iii) a nucleic acid sequence as represented in SEQ ID NO: 9;
iv) a nucleic acid sequence as represented in SEQ ID NO:11;
v) a nucleic acid sequence as represented in SEQ ID NO:13;
vi) a nucleic acid sequence as represented in SEQ ID NO:15;
vii) a nucleic acid sequence as represented in SEQ ID NO:17;
viii) a nucleic acid sequence as represented in SEQ ID NO:19; or
   a nucleic acid molecule comprising a nucleic sequence that hybridizes under stringent hybridization conditions to SEQ ID NO:5-SEQ ID NO: 19 and which encodes a polypeptide that has granulocyte colony stimulating factor receptor modulating activity.

In a preferred embodiment of the invention said nucleic acid molecule encodes a polypeptide that has agonist activity.

In a preferred embodiment of the invention said nucleic acid molecule encodes a polypeptide that has antagonist activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecule comprises or consists of a nucleic acid sequence as represented in SEQ ID NO: 5.

In a preferred embodiment of the invention said nucleic acid molecule comprises or consists of a nucleic acid sequence as represented in SEQ ID NO: 7.

In a preferred embodiment of the invention said nucleic acid molecule comprises or consists of a nucleic acid sequence as represented in SEQ ID NO: 9.

In a preferred embodiment of the invention said nucleic acid molecule comprises or consists of a nucleic acid sequence as represented in SEQ ID NO: 11.

In a preferred embodiment of the invention said nucleic acid molecule comprises or consists of a nucleic acid sequence as represented in SEQ ID NO: 13.

In a preferred embodiment of the invention said nucleic acid molecule comprises or consists of a nucleic acid sequence as represented in SEQ ID NO: 15.

In a preferred embodiment of the invention said nucleic acid molecule comprises or consists of a nucleic acid sequence as represented in SEQ ID NO: 17.

In a preferred embodiment of the invention said nucleic acid molecule comprises or consists of a nucleic acid sequence as represented in SEQ ID NO: 19.

According to an aspect of the invention there is provided a polypeptide encoded by the nucleic acid according to the invention.

According to a further aspect of the invention there is provided a polypeptide comprising or consisting of an amino acid sequence selected from the group consisting of: SEQ ID NO: 6, 8, 10, 12, 14, 16, 18, 20, 25, 26, 27, 28, 29 or 30.

In a preferred embodiment of the invention said polypeptide has agonist activity.

In an alternative preferred embodiment of the invention said polypeptide has antagonist activity.

According to an aspect of the invention there is provided a homodimer consisting of two polypeptides wherein each of said polypeptides comprises:
i) a first part comprising granulocyte colony stimulating factor, or a receptor binding domain thereof, optionally linked by a peptide linking molecule to
ii) a second part comprising the cytokine homology binding domain or part thereof, of the granulocyte colony stimulating factor receptor.

In a preferred embodiment of the invention said homodimer comprises two polypeptides comprising or consisting of SEQ ID NO: 6.

In a preferred embodiment of the invention said homodimer comprises two polypeptides comprising or consisting of SEQ ID NO: 8.

In a preferred embodiment of the invention said homodimer comprises two polypeptides comprising or consisting of SEQ ID NO: 10.

In a preferred embodiment of the invention said homodimer comprises two polypeptides comprising or consisting of SEQ ID NO: 12.

In a preferred embodiment of the invention said homodimer comprises two polypeptides comprising or consisting of SEQ ID NO: 14.

In a preferred embodiment of the invention said homodimer comprises two polypeptides comprising or consisting of SEQ ID NO: 16.

In a preferred embodiment of the invention said homodimer comprises two polypeptides comprising or consisting of SEQ ID NO: 18.

In a preferred embodiment of the invention said homodimer comprises two polypeptides comprising or consisting of SEQ ID NO: 20.

In a preferred embodiment of the invention said homodimer comprises two polypeptides comprising or consisting of SEQ ID NO: 25.

In a preferred embodiment of the invention said homodimer comprises two polypeptides comprising or consisting of SEQ ID NO: 26.

In a preferred embodiment of the invention said homodimer comprises two polypeptides comprising or consisting of SEQ ID NO: 27.

In a preferred embodiment of the invention said homodimer comprises two polypeptides comprising or consisting of SEQ ID NO: 28.

In a preferred embodiment of the invention said homodimer comprises two polypeptides comprising or consisting of SEQ ID NO: 29.

In a preferred embodiment of the invention said homodimer comprises two polypeptides comprising or consisting of SEQ ID NO: 30.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

In a preferred embodiment of the invention said cell is stably transfected or transformed.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringers solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from a condition that would benefit from administration of a granulocyte colony stimulating factor agonist comprising administering an effective amount of at least one polypeptide according to the invention.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

In a preferred method of the invention said condition is neutropenia.

According to a further aspect of the invention there is provided a method to stimulate haematopoietic progenitor cell proliferation and/or differentiation in a human subject comprising administering an effective amount of at least one polypeptide according to the invention.

In a preferred method of the invention said method is an in vitro method.

In an alternative preferred method of the invention said method is an in vivo method.

In a preferred method of the invention following stimulation of haematopoietic progenitor cells bone marrow is harvested from said human subject and used for haematopoietic progenitor cell transplantation.

Preferably said harvested bone marrow is administered to a human subject in need of bone marrow transplantation.

According to an aspect of the invention there is provided the use of a polypeptide according to the invention for the manufacture of a medicament for the treatment of neutropenia.

In a further preferred embodiment of the invention said polypeptide is administered at two day intervals; preferably said polypeptide is administered at weekly, 2 weekly or monthly intervals.

According to a further aspect of the invention there is provided the use of an effective amount of a polypeptide according to the invention in the manufacture of a medicament for the stimulation of haematopoietic progenitor cell of proliferation and/or differentiation in a human subject.

According to a further aspect of the invention there is provided a monoclonal antibody that binds the polypeptide or dimer according to the invention.

Preferably said monoclonal antibody is an antibody that binds the polypeptide or dimer but does not specifically bind granulocyte colony stimulating factor or granulocyte colony stimulating factor receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide or dimer according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

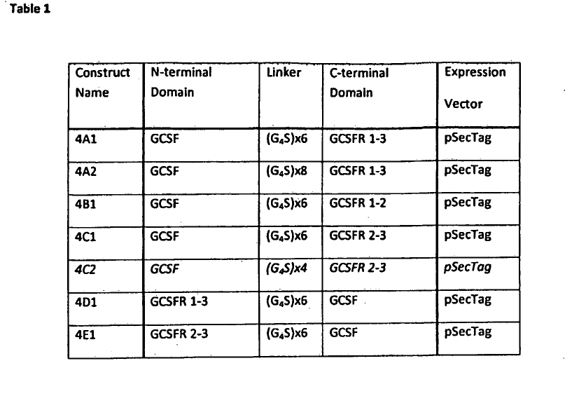

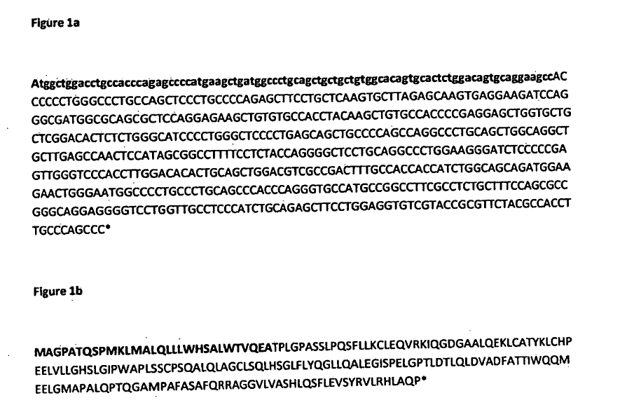

FIG. 1a nucleic acid sequence encoding GCSF expressed in a mammalian cell line. Signal sequence is shown in bold and lower case. *refers to stop codon. Nucleotide length=522 bp, (not including signal sequence); FIG. 1b amino acid sequence length=174aa (not including signal sequence);

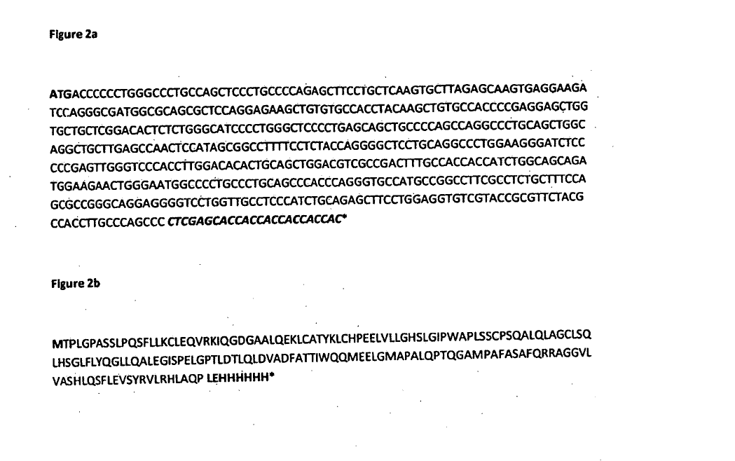

FIG. 2a nucleic acid sequence encoding GCSF expressed in E. coli (pET21a (+)), with 6× Histidine tag; ATG start codon in bold. *refers to stop codon. Letters in bold italics refer to excess sequence at 5′ end due to Hist-tag; FIG. 2b mature amino acid sequence length=182aa (not including methionine start);

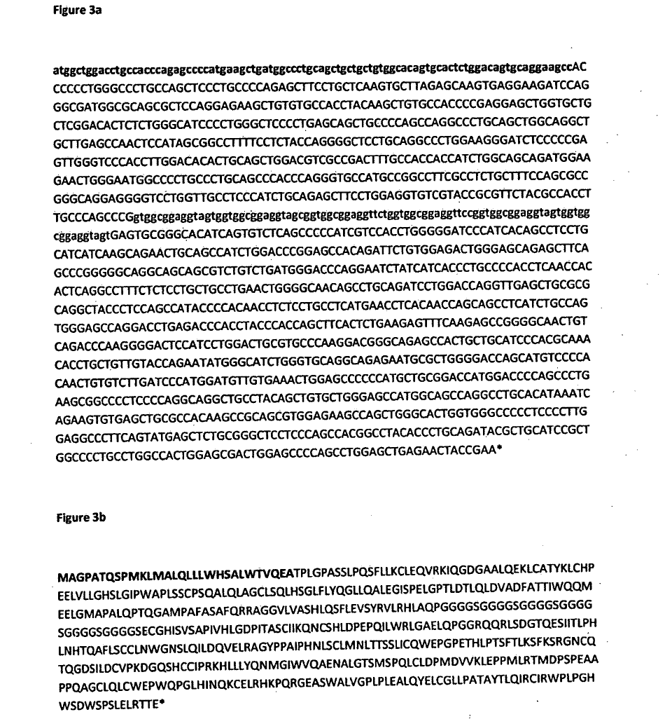

FIG. 3a nucleic acid sequence encoding GCSF-L6-GCSFrEC (1-3): contains GCSF linked via G4Sx6 to GCSF extracellular receptor domains 1-3 (Ig, BN and BC). *refers to stop codon. Signal sequence in bold and lower case; FIG. 3b amino acid sequence length=511aa (not including signal sequence);

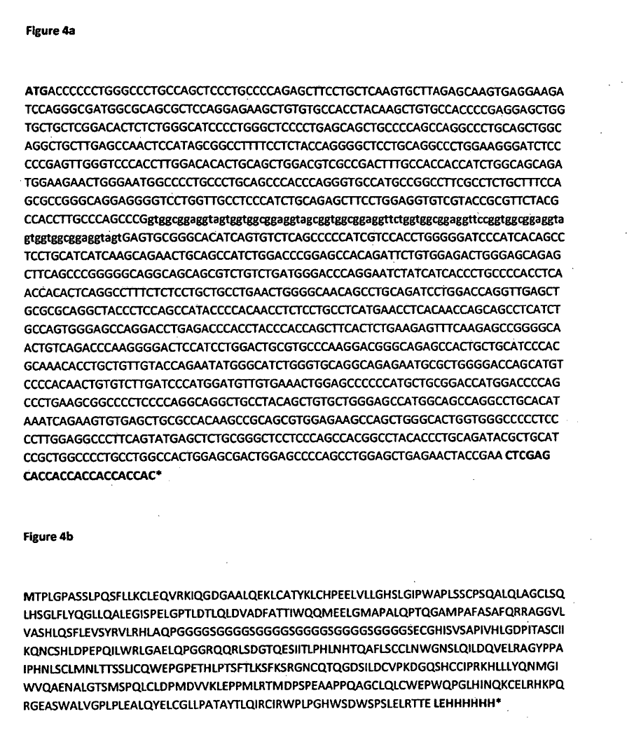

FIG. 4a nucleic acid sequence encoding GCSF-L6-GCSFrEC (1-3) expressed in E. coli: contains GCSF linked via G4Sx6 to GCSF extracellular receptor domains 1-3 (Ig, BN and BC). *refers to stop codon; ATG start codon in bold. *refers to stop codon. Letters in bold italics refer to excess sequence at 5′ end due to Hist-tag; FIG. 4b amino acid sequence length=519aa (not including Methionine start);

FIG. 5a nucleic acid sequence encoding GCSF-L8-GCSFrEC (1-3) expressed in a mammalian cell line: contains GCSF linked via G4Sx8 to GCSF extracellular receptor domains 1-3 (Ig, BN and BC). *refers to stop codon. Signal sequence in bold and lower case; FIG. 5b amino acid sequence length=521aa (not including signal sequence)

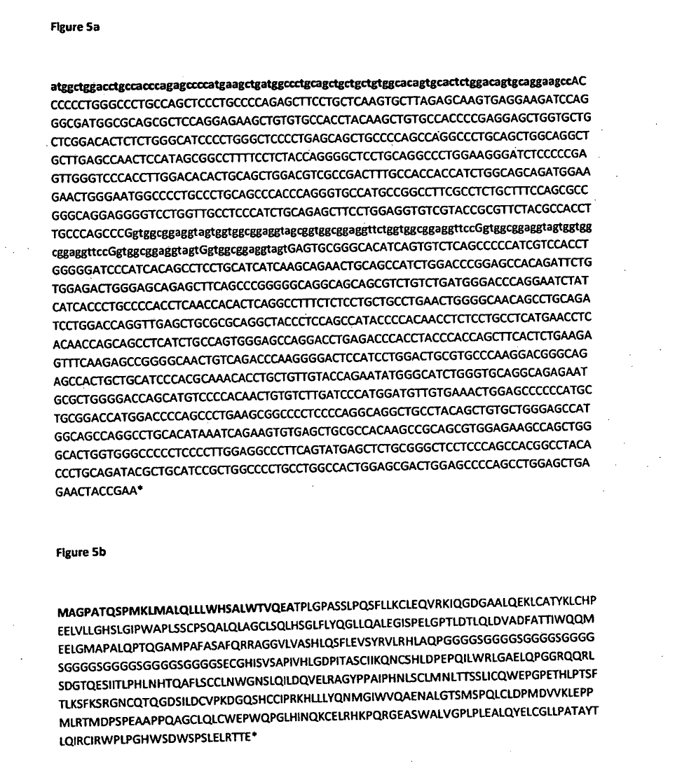

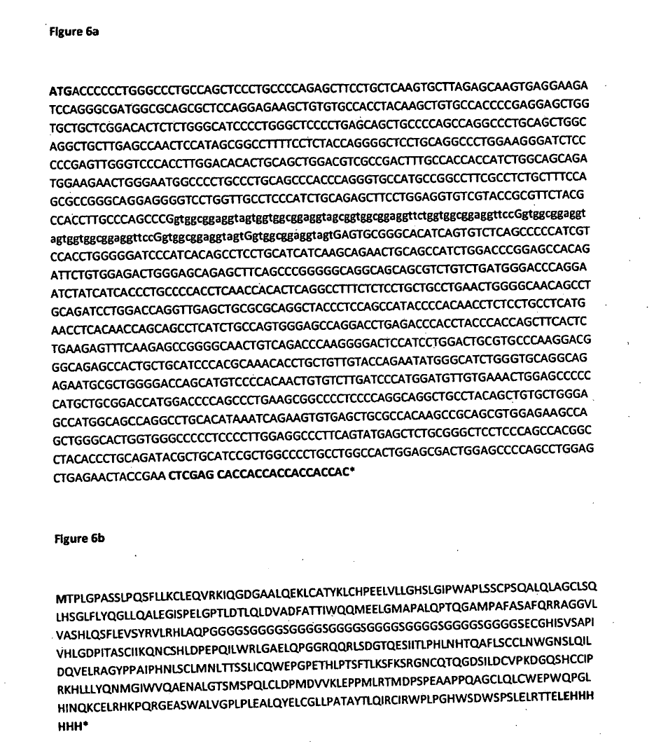

FIG. 6a nucleic acid sequence encoding GCSF-L8-GCSFrEC (1-3) expressed in E. coli contains GCSF linked via G4Sx8 to GCSF extracellular receptor domains 1-3 (Ig, BN and BC). *refers to stop codon; FIG. 6b amino acid sequence length=529aa (not including Methionine start)

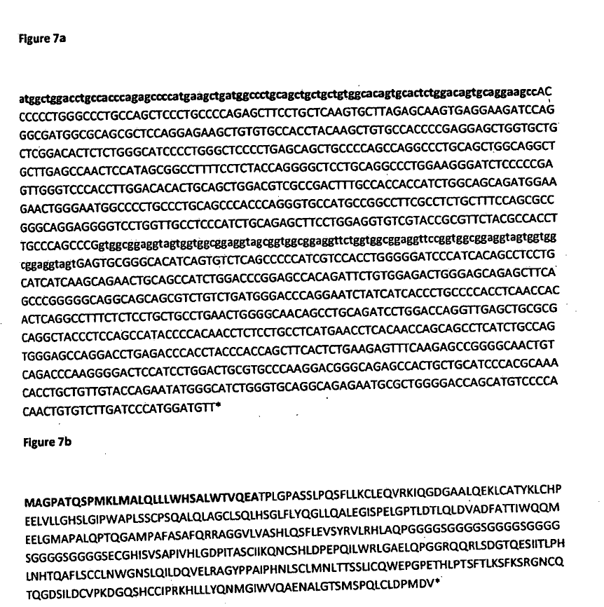

FIG. 7a nucleic acid encoding GCSF-L6-GCSFrEC (1-2): contains GCSF linked via G4Sx6 to GCSF extracellular receptor domains 1-2 (Ig and BN). *refers to stop codon. Signal sequence in bold and lower case; FIG. 7b amino acid sequence length=404aa (not including signal sequence)

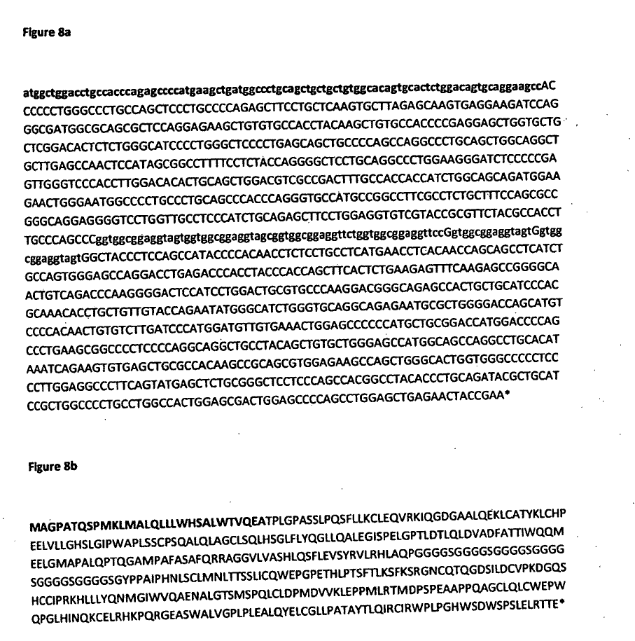

FIG. 8a nucleic acid encoding GCSF-L6-GCSFrEC (2-3): contains GCSF linked via G4Sx6 to GCSF extracellular receptor domains 2-3 (BN and BC). *refers to stop codon. Signal sequence in bold and lower case; FIG. 8b amino acid sequence length=416aa (not including signal sequence)

FIG. 9a nucleic acid encoding GCSFrEC (1-3)-L6-GCSF: contains GCSFrEC (domains 1-3) linked via G4Sx6 to GCSF. *refers to stop codon. Signal sequence in bold and lower case; FIG. 9b amino acid sequence length=511aa (not including signal sequence)

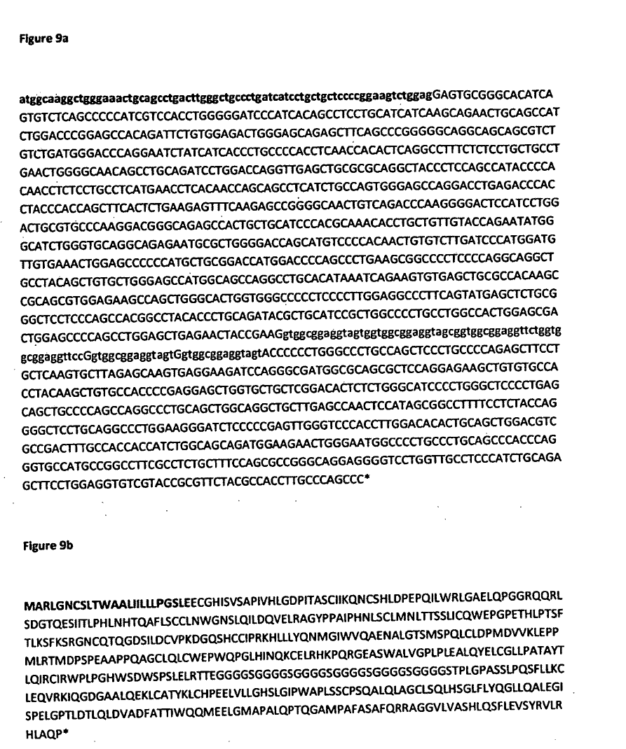

FIG. 10a nucleic acid encoding GCSFrEC (2-3)-L6-GCSF expressed in a mammalian cell line: contains GCSFrEC (domains 2-3) linked via G4Sx6 to GCSF. *refers to stop codon. Signal sequence in bold and lower case; FIG. 10b amino acid sequence length=416aa (not including signal sequence)

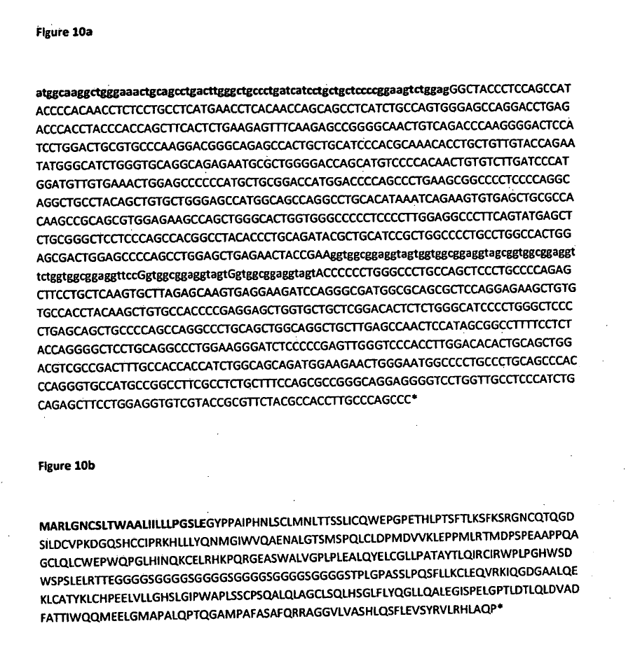

FIG. 11a nucleic acid sequence encoding GCSFrEC expressed in a mammalian cell line: contains GCSF receptor extracellular domains 1-3. Signal sequence in is bold and lower case. *refers to stop codon; FIG. 11b amino acid sequence length=307aa (not including signal sequence); and

FIG. 12a nucleic acid sequence GCSFrEC (Extracellular domains 1-3) expressed in pET21a (+) with 6× histidine tag (*refers to stop codon, letters in bold refer to extra XhoI restriction site and 6× Hist-tag); FIG. 12b amino acid sequence length=315aa (not including Met);

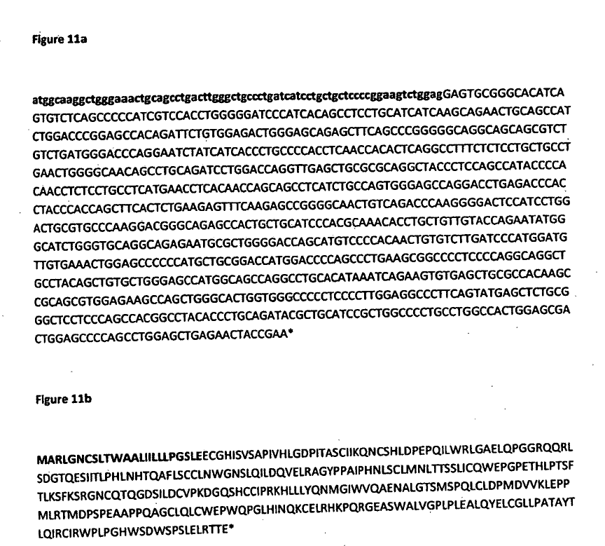

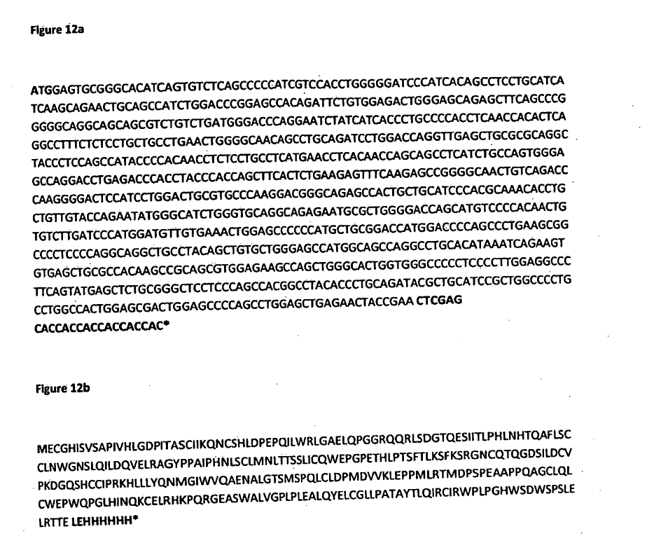

FIG. 13 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites); and

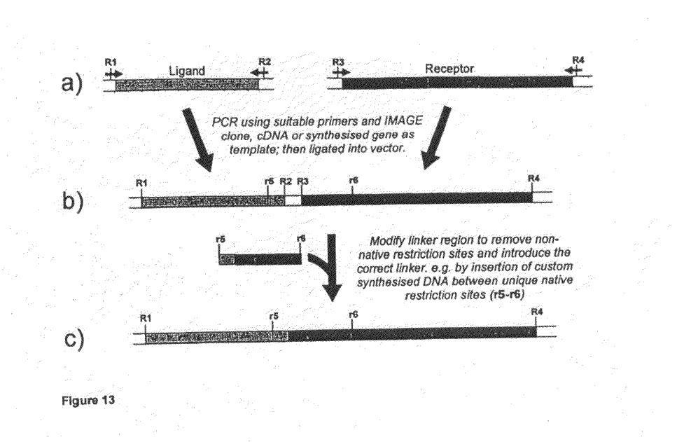

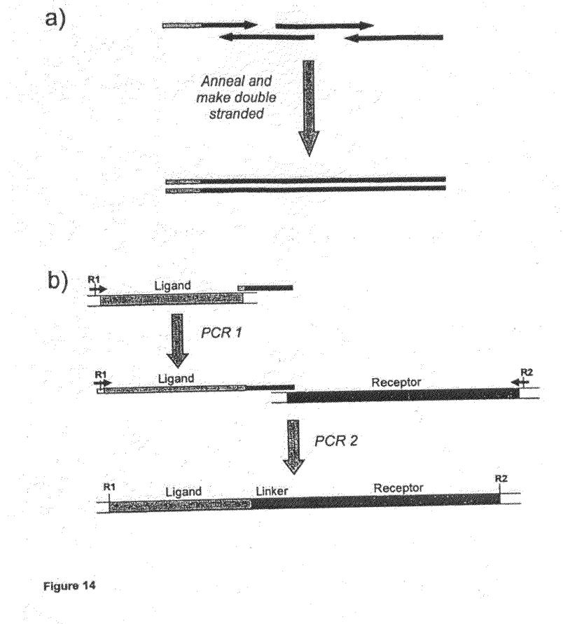

FIG. 14 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

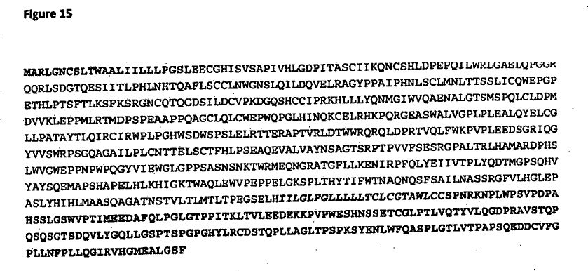

FIG. 15 is the complete amino acid sequence of granulocyte colony stimulating factor receptor;

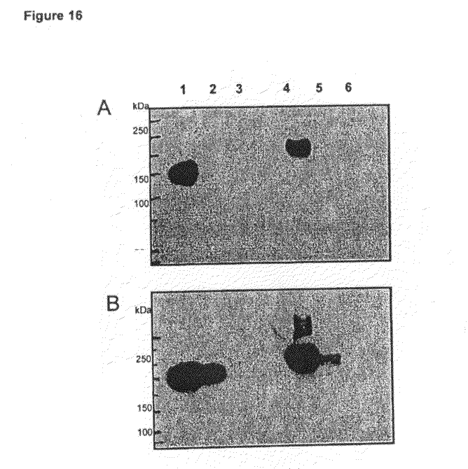

FIG. 16 illustrates western blot analysis of CHO flpIn stable cell lines expressing GCSF chimeric constructs Lane 1=4A1; Lane 2=4D1; Lane 3=Mock media; Lane 4=4A1; Lane 5=4D1; lane 6=Mock media; A=Non reducing conditions; B=reducing conditions. Both 4A1 and 4D1 run between 75 and 100 kDa. GCSF runs between 37 and 45 kDa as expected for the glycosylated protein;

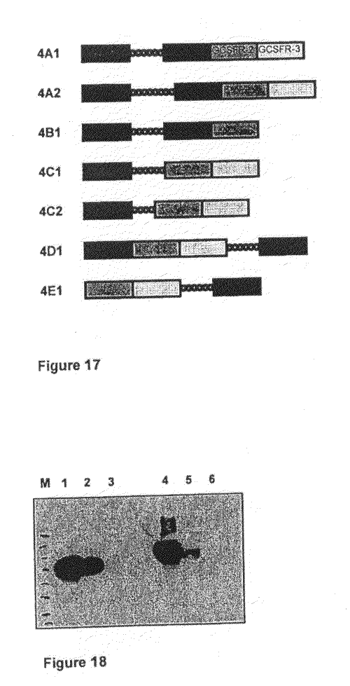

FIG. 17 is a schematic diagram of the GCSF LR-fusion constructs;

FIG. 18 is an immuno-blot analysis of CHO Flp-In stable cell lines expressing 4A1 and 4D1 constructs. Lane M=Markers (at 250, 150, 100, 75, 50, 37, 25 and 20 kDa); Lane 1=4A1; Lane 2=4D1; Lane 3=Mock media; Lane 4=4A1; Lane 5=4D1; lane 6=Mock media. Lanes 1-3=reducing conditions and lanes 4-6=non reducing conditions;

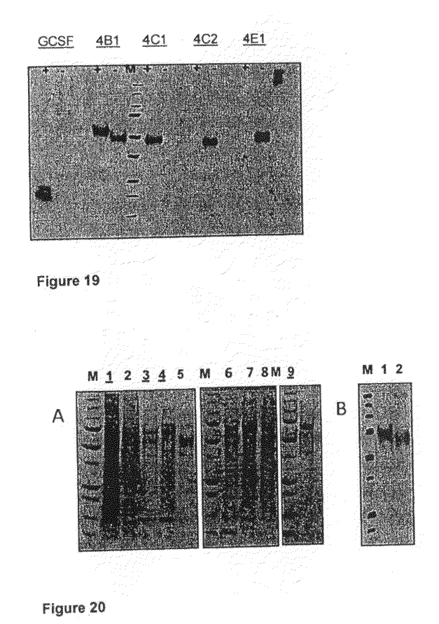

FIG. 19 is an immuno-blot analysis of CHO Flp-In stable cell lines expressing GCSF, 4B1, 4C1, 4C2 and 4E1 constructs. Lane M=Markers (at 250, 150, 100, 75, 50, 37, 25, and 15 kDa); −=no DTT (non-reduced); +=with DTT (reduced);

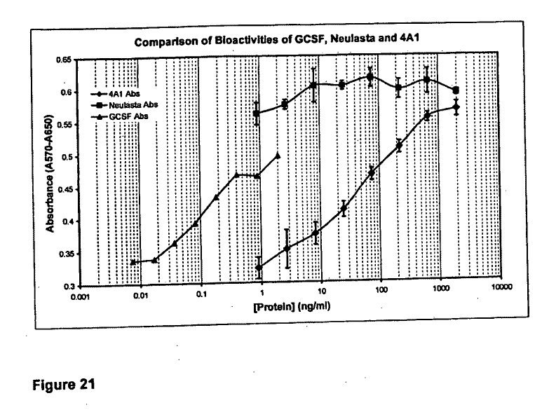

FIG. 20 (A) Coomassie stained gels of the 4A1 purification steps. Lane M=Markers (at 250, 150, 100, 75, 50, 37, 25, 20 and 15 kDa); Lane 1=Crude media 10× concentrate; Lane 2=pH4.5 precipitation pellet, Lane 3=pH 4.5 precipitation supernatant, Lane 4=pH 5.5 SP unbound, Lane 5=pH 5.5 SP bound, Lane 6-8=fractions 4 to 6 from the pH 8.0 Q column, Lane 9=50% ammonium sulphate precipitation pellet. (B) Immuno-blot of the purified 4A1. Lane M=Markers (as above), Lane 1=4A1 (with OTT; reduced), Lane 2=4A1 (no OTT; non-reduced); and

FIG. 21 illustrates an in vitro bioassay measuring activity of GCSF, Neulasta and the GCSF LR fusion 4A1.

### MATERIALS AND METHODS

### In Vitro Testing

In vitro methods to test the activity of the GCSF fusion polypeptides are known in the art. For example, it is known to harvest blood, bone and spleen cells from an animal to test the colony forming ability of GCSF (see Liu et al Blood, 15 May 2000; 95(10), p3025-3031). In addition, the use of cells that express GCSFR for example M-NFS-60 cells and that are stimulated to proliferate as measured by ³H— thymidine is known.

### In Vivo Testing

Various animal models are available to test the activity of GCSF. For example, Harada et al (Nature Medicine 11: 305-311, 2005) describes a mouse model of myocardial infarction in which the effects of GCSF were tested to monitor the effects of administered recombinant GCSF on cardiac function.

### Immunological Testing

Immunoassays that measure the binding of granulocyte colony stimulating factor to polyclonal and monoclonal antibodies are known in the art. Commercially available granulocyte colony stimulating factor antibodies are available to detect granulocyte colony stimulating factor in samples and also for use in competitive inhibition studies. For example see, http://www.scbt.com/index.html, Santa Cruz Biotechnology Inc.

### Recombinant Production of Fusion Proteins

The components of the fusion proteins were generated by PCR using primers designed to anneal to the ligand or receptor and to introduce suitable restriction sites for cloning into the target vector (FIG. 13a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 13b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 13c).

Alternatively, the linker with flanking sequence, designed to anneal to the ligand or receptor domains of choice, was initially synthesised by creating an oligonucleotide duplex and this processed to generate double-stranded DNA (FIG. 14a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 14b).

### Expression and Purification of Fusion Proteins

Expression was carried out in a suitable system (e.g. mammalian CHO cells, E. coli,) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the LR-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

### Characterisation of LR Fusions

Denaturing PAGE, native PAGE gels and western blotting were used to analyse the fusion, polypeptides and western blotting performed with antibodies non-conformationally sensitive to the LR-fusion. Native solution state molecular weight information can be obtained from techniques such as size exclusion chromatography using a Superose G200 analytical column and analytical ultracentrifugation.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

### Construction of Chimeric Clones

GCSF extracellular receptor domains 1-3 were PCR'd directly from a clone obtained from the Image Consortium and cloned into the mammalian expression vector pSecTag-link. Both genes for 4A1 (FIG. 3a; FIGS. 3b) and 4D1 (FIG. 9a; FIG. 9b) were constructed using gene synthesis and cloned into the mammalian expression vector pSecTag-link to form pGCSFsecTag-4A1 and 4A5

### Mammalian Stable Expression of GCSF and Chimeric Clones

A mammalian expression system has been established using a modified Invitrogen vector pSecTag-V5/FRT-Hist. This system allows for the rapid generation of stable clones into specific sites within the host genome for high expression. This can be used with either secreted or cytoplasmic expressed proteins. Flp-In host cell lines (flp-In CHO) have a single Flp recombinase target (FRT) site located at a transcriptionally active genomic locus. Stable cell lines are generated by co-transfection of vector (Containing FRT target site) and pOG44 (a [plasmid that transiently expresses lip recombinase) into Flp-In cell line. Selection is with Hygromycin B. There is no need for clonal selection since integration of DNA is directed. Culturing Flp-In Cell lines: followed manufactures instruction using basic cell culture techniques.

### Stable Transfection of CHO Flp-In Cells Using Fugene-6

The day before transfection CHO Flp-In cells were seeded at 6×10E5 per 100 mm petri dish in a total volume of 10 ml of Hams F12 media containing 10% (v/v) Fetal Calf Serum, 1% Penicillin/streptomycin and 4 mM L-glutamine. The next day added 570 μl of serum free media (containing no antibiotics) to a 1.5 ml polypropylene tube. 30 μl of fugene-6 was then added and mixed by gentle rolling. A separate mix of plasmids was set up for each transfection which combined 2 μg plasmid of interest with 18 μg pOG44 (plasmid contains recombinase enzyme necessary for correct integration of plasmid into host genome). Control plate received no plasmid. This was mixed with fugene-6 by gentle rolling, left @ RmT for 15 minutes, then applied drop-wise to the surface of the each petri dish containing CHO Flp-In cells in F12 media+10% FCS. The plates were gently rolled to ensure good mixing and left for 24 hrs @ 37° C./5% CO2. The next day media was exchanged for selective media containing hygromycin B @ 600 ug/ml. Cells were routinely kept at 60% confluency or less. Cells were left to grow in the presence of 600 ug/ml hygromycin B until control plate cells (non transfected cells) had died (i.e. no hygromycin resistance).

### SDS-PAGE Analysis and Western Blotting

Stable transfected CHO Flp-In cell lines were grown in 75 cm2 flasks for approximately 3-4 days, at which point samples were taken for analysis. Samples were mixed with an equal volume of Laemmli loading buffer in the presence and absence of 25 mM DTT and boiled for 5 minutes. Samples were separated on a 4-20% (w/v) bis-acrylamide gel and transferred to a PVDF membrane (FIG. 16). After blocking in 5% (w/v) Milk protein in PBS-0.05% (v/v) Tween 20, sample detection was carried out using a specific anti-GCSF antibody together with a Horse Radish Peroxidase (HRP) conjugated secondary antibody. Visualisation was by chemiluminesence on photographic film using an HRP detection kit.

### Construction of LR-Fusions

4A1 and 4D1 were gene synthesised (Genecust, France) and inserted into the mammalian expression vector pSegTag. 4B1 and 4E1 were generated by using PCR to truncate the 4A1 and 4D1 genes, respectively. 4A2, 4C1 and 4C2 were generated by synthesising a primer duplex for the linker region and using PCR to extend this into the GCSF and GCSFR sequences. 4A2 was not synthesised due to the failure of the PCRs to extend the (G4S)₈ linker sequence into the full length gene. 4C2 was generated as a by-product of the synthesis of 4C1. A schematic of constructs for GCSF-LR fusion protein is shown in FIG. 17.

### Expression of LR-Fusions

A mammalian expression system has been established using a modified Invitrogen vector pSecTag-V5/FRT-Hist. This vector is used in Invitrogen's Flp-In system to direct integration of the target gene into the host cell line, allowing rapid generation of stable clones into specific sites within the host genome for high expression.

Culturing Flp-In Cell lines: followed manufactures instruction using basic cell culture techniques.

Stable cell lines were generated in 6-well plates using Fugene-6 as the transfection reagent. The CHO Flp-In cells were co-transfected with the expression vector and pOG44, a plasmid that expresses flp recombinase an enzyme which causes the recombination of the LR-fusion gene into a “hot-spot” of the cell chromosome. Hygromycin B was used to select for cells with positive recombinants.

Once the stable cell lines had been established they were grown on 75 cm² culture plates, at a confluency of 50-70% the media was changed to serum free media. The cultures were incubated for a further 2-4 days after which media samples were taken. These were run on 13% SDS-PAGE gels and transferred to PVDF membrane for immuno-blotting. After blocking in 5% (w/v) milk protein in PBS+0.05% (v/v) Tween 20, sample detection was carried out using a specific anti-GCSF antibody together with a Horse Radish Peroxidase (HRP) conjugated secondary antibody. Visualisation was by chemiluminesence on photographic film using an HRP detection kit. The immuno-blots showing the expression of the LR-fusions are shown in FIGS. 18 and 19.

### Purification of LR-Fusions

The purification methodology for GCSF-LR is detailed below and shown in FIG. 20.
a) Protease inhibitors were added to a 10× concentrated media from cells expressing 4A1.
b) The 10× concentrated media was dialysed against 50 mM TRIS, 1 mM EDTA, pH 8.0 for 2-4 hours.
c) The protein and dialysis tubing from (2) was transferred to a solution of 10 mM TRIS, 1 mM EDTA, pH 8.0 for 16 hours (overnight).
d) 0.25 volumes of 0.1M acetate buffer, pH 4.5 was added. The pH was checked using pH indicator strips and more 0.1M acetate buffer, pH 4.5 added in 0.1 ml aliquots until the pH reached 4.5.
e) This was then incubated on ice for 2 hours with periodic mixing.
f) Centrifugation was performed to remove the precipitate and the supernatant transferred to a new tube.
g) 0.5M/0.1M NaOH was added to the supernatant in 0.1 ml aliquots until the pH changed to 5.5—analysed pH using pH indicator strips.
h) The solution was then loaded onto a 5 ml SP FF column pre-equilibrated with 25 mM acetate buffer, pH 5.5. The unbound protein was collected.
i) The unbound fraction was dialysed against 25 mM TRIS, 1 mM EDTA, pH 8.0 for 16 hours (overnight).
j) The dialysed sample was loaded onto a 5 ml Q FF column pre-equilibrated with 25 mM TRIS, pH 8.0.
k) The column was washed with 20 column volumes of 25 mM TRIS, pH 8.0.
l) Protein was then eluted off the column using a 0-1M NaCl gradient over 20 column volumes, collecting 1 column volume fractions. [4A1 is eluted in fraction 4-7 on a 5 ml column].
m) Fractions containing >70% pure 4A1 were pooled and incubated on ice.
n) An equal volume of ice cold saturated ammonium sulphate solution was added to give 50% ammonium sulphate saturation. Incubated on ice for 2-3 hours.
o) The protein sample was then centrifuged to pellet precipitated protein.
p) The pellet was re-suspended in PBS and dialysed against PBS.
q) The protein sample was then analysed.

### In Vitro Bioassay

### Cell Preparation

AML-193 cells (ATCC, Batch No. 3475266) were removed from liquid nitrogen storage and placed into a 37° C. waterbath for 2 min. The contents of the vial were then transferred to a 15 ml tube containing 9 ml of culture medium (5% FBS, 4 mM L-glutamine, 100 U/ml penicillin, 100 μg/ml streptomycin, 5 ng/ml GM-CSF, 5 μg/ml insulin, 5 μg/ml transferrin in Iscove's modified Dulbecco's medium). Cells were centrifuged for 5 min at 123×g, the cell pellet was resuspended in culture medium and cell density adjusted to 2.3×10⁵ cells/ml.

### Cell Culture

Cells were cultured in CO₂ incubator (5% CO₂, 37° C.) in culture medium at a density of 3×10⁵−2×10⁶ cells/ml. Passages were performed twice a week ensuring cell density did not exceed 2.5×10⁶ cells/ml. Cell viability was assessed by trypan blue exclusion. Prior to assay cells were washed 3 times with PBS by spinning for 5 min at −125×g. The pellet was then reconstituted in assay medium (5% FBS, 4 mM L-glutamine, 100 U/ml penicillin, 100 μg/ml streptomycin, 5 μg/ml insulin, 5 μg/ml transferrin in Iscove's modified Dulbecco's medium) and cell density was adjusted to 5×10⁵ cells/ml.

### Standard/Sample Preparation

Appropriate dilutions of GCSF protein solution of 4A1 were made in PBS (1% BSA) for bioactivity testing. GCSF (international standard, NIBSC, Batch No 88/502) were reconstituted in 50% solution of phosphate buffered saline and water (both sterile) to a concentration of 10 ng/ml (10000 IU/ml), divided into 40 μl aliquots and stored at −80° C. On each day of assay 1 vial was removed from the freezer and working concentrations were prepared.

The in vitro bioactivity for GCSF-LR (4A1) is shown in FIG. 21.

1. (canceled)

2. A fusion polypeptide comprising: the amino acid sequence of granulocyte colony stimulating factor polypeptide, or active part thereof linked, directly or indirectly, to at least one cytokine binding domain of the granulocyte colony stimulating factor receptor polypeptide.

3. (canceled)

4. A fusion polypeptide according to claim 2 wherein said polypeptide further comprises an immunoglobulin-like domain.

5. A fusion polypeptide according to claim 2 wherein said polypeptide includes at least one fibronectin III domain.

6. A fusion polypeptide according to claim 2 wherein said polypeptide comprises a sequence selected from the group consisting of:
(a) amino acid residues 97-201 as represented in SEQ ID NO: 31;
(b) amino acid residues 202-313 of SEQ ID NO: 31;
(c) amino acid residues 97-313 of SEQ ID NO: 31; and
(d) amino acid residues 1-97 of SEQ ID NO: 31.

7-11. (canceled)

12. A fusion polypeptide according to claim 2 wherein granulocyte colony stimulating factor polypeptide is linked to the binding domain of the granulocyte colony stimulating factor receptor polypeptide by a peptide linker.

13. A fusion polypeptide according to claim 12 wherein said peptide linking molecule comprises at least one copy of the peptide Gly Gly Gly Gly Ser (SEQ ID NO: 33).

14-15. (canceled)

16. A fusion polypeptide according to claim 2 wherein said polypeptide does not comprise a peptide linking molecule and is a direct fusion of granulocyte colony stimulating factor polypeptide and the binding domain of granulocyte colony stimulating factor receptor polypeptide.

17. An isolated nucleic acid molecule comprising a nucleic acid sequence selected from:
i) a nucleic acid sequence as represented in SEQ ID NO:5;
ii) a nucleic acid sequence as represented in SEQ ID NO 7:;
iii) a nucleic acid sequence as represented in SEQ ID NO: 9;
iv) a nucleic acid sequence as represented in SEQ ID NO:11;
v) a nucleic acid sequence as represented in SEQ ID NO:13;
vi) a nucleic acid sequence as represented in SEQ ID NO:15;
vii) a nucleic acid sequence as represented in SEQ ID NO:17;
viii) a nucleic acid sequence as represented in SEQ ID NO:19; and
ix) a nucleic acid sequence that hybridizes under stringent hybridization conditions to SEQ ID NO:5-SEQ ID NO: 19 and which encodes a polypeptide that has granulocyte colony stimulating factor receptor modulating activity.

18-27. (canceled)

28. An isolated polypeptide encoded by the nucleic acid according to claim 17.

29. An isolated polypeptide comprising an amino acid sequence selected from the group consisting of: SEQ ID NO: 6, 8, 10, 12, 14, 16, 18, 20, 25, 26, 27, 28, 29 and 30.

30-31. (canceled)

32. A homodimer consisting of two polypeptides wherein each of said polypeptides comprises:
i) a first part comprising granulocyte colony stimulating factor, or a receptor binding domain thereof; and
ii) a second part comprising the cytokine homology binding domain or part thereof, of the granulocyte colony stimulating factor receptor.

33. A homodimer according to claim 32 wherein said homodimer comprises two polypeptides comprising a sequence selected from the group consisting of: SEQ ID NO: 6, 8, 10, 12, 14, 16, 18, 20, and 25-30.

34-46. (canceled)

47. A vector comprising a nucleic acid molecule according to claim 17.

48. A cell transfected or transformed with a nucleic acid molecule according to claim 17.

49-50. (canceled)

51. A pharmaceutical composition comprising a polypeptide according to according to claim 2 and an excipient or carrier.

52. (canceled)

53. A method to treat a human subject suffering from a condition that would benefit from administration of a granulocyte colony stimulating factor polypeptide comprising administering an effective amount of at least one polypeptide according to claim 2.

54-59. (canceled)

60. A method according to claim 53 wherein said condition is neutropenia.

61. A method to stimulate haematopoietic progenitor cell proliferation and/or differentiation in a human subject comprising administering an effective amount of at least one polypeptide according to claim 2.

62-74. (canceled)


SEQUENCE LISTING (33 sequences)

SEQ ID NO: 1 - DNA, 612 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 2 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - DNA, 549 bp, Homo sapiens
atgacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccctcgagcaccaccaccaccaccac

SEQ ID NO: 4 - protein, 176 aa, Artificial
  bacterial expressed GCSF
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPLE

SEQ ID NO: 5 - DNA, 1623 bp, Artificial
  GCSF/GCSFR fusion protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggtagtgagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 6 - protein, 541 aa, Artificial [sequence omitted]

SEQ ID NO: 7 - DNA, 1536 bp, Artificial
  GCSF/GCSFR fusion protein
atgacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggtagtgagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 8 - protein, 511 aa, Artificial
  GCSF/GCSFR fusion protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSGGGGSECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTE

SEQ ID NO: 9 - DNA, 1653 bp, Artificial
  GCSF/GCSFR fusion protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggttccggtggcggaggtagtggtggcggaggtagtgagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 10 - protein, 551 aa, Artificial [sequence omitted]

SEQ ID NO: 11 - DNA, 1566 bp, Artificial
  GCSF/GCSFR fusion protein
atgacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggttccggtggcggaggtagtggtggcggaggtagtgagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 12 - protein, 521 aa, Artificial [sequence omitted]

SEQ ID NO: 13 - DNA, 1302 bp, Artificial
  GCSF/GCSFR fusion protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggtagtgagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgtt

SEQ ID NO: 14 - protein, 434 aa, Artificial
  GCSF/GCSFR fusion protein
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSGGGGSECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDV

SEQ ID NO: 15 - DNA, 1338 bp, Artificial
  GCSF/GCSFR fusion protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtggtggcggaggtagtggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 16 - protein, 446 aa, Artificial
  GCSF/GCSFR fusion protein
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSGGGGSGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTE

SEQ ID NO: 17 - DNA, 1605 bp, Artificial
  GCSF/GCSFR fusion protein
atggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaaggtggcggaggtagtggtggcggaggtagcggtggcggaggttctggtggcggaggttccggtggcggaggtagtggtggcggaggtagtacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 18 - protein, 535 aa, Artificial
  GSCF/GCSFR fusion protein
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTEGGGGSGGGGSGGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 19 - DNA, 1320 bp, Artificial
  GCSF/GCSFR fusion protein
atggcaaggctgggaaactgcagcctgacttgggctgccctgatcatcctgctgctccccggaagtctggagggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaaggtggcggaggtagtggtggcggaggtagcggtggcggaggttctggtggcggaggttccggtggcggaggtagtggtggcggaggtagtacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 20 - protein, 440 aa, Artificial
  GCSF/GCSFR fusion protein
MARLGNCSLTWAALIILLLPGSLEGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTEGGGGSGGGGSGGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 21 - DNA, 993 bp, Homo sapiens
atggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 22 - protein, 331 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTE

SEQ ID NO: 23 - DNA, 924 bp, artificial
  bacterial expressed GCSF extracellular domain
atggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaa

SEQ ID NO: 24 - protein, 307 aa, Artificial
  bacterial expressed GCSF
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTE

SEQ ID NO: 25 - protein, 511 aa, Artificial; identical to SEQ ID NO: 8
  GCSF/GCSFR fusion protein

SEQ ID NO: 26 - protein, 521 aa, Artificial [sequence omitted]

SEQ ID NO: 27 - protein, 404 aa, Artificial
  GCSF/GCSFR fusion protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSGGGGSECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDV

SEQ ID NO: 28 - protein, 416 aa, Artificial
  GCSF/GCSFR fusion protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSGGGGSGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTE

SEQ ID NO: 29 - protein, 511 aa, Artificial
  GCSF/GCSFR fusion protein
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTEGGGGSGGGGSGGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 30 - protein, 416 aa, Artificial
  GCSF/GCSFR fusion protein
GYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTEGGGGSGGGGSGGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 31 - protein, 307 aa, Homo sapiens; identical to SEQ ID NO: 24

SEQ ID NO: 32 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 33 - protein, 5 aa, Artificial Sequence
  Made in a lab
GGGGS